(19) United States
(12) Patent Application Publication
(10) Pub. No.: US 2020/0155478 A1
(43) Pub. Date: May 21, 2020

(54) XIBORNOL FOR USE IN THE TREATMENT OF ACNE VULGARIS

(71) Applicant: ABIOGEN PHARMA S.P.A., Ospedaletto (IT)

(72) Inventor: Silvia Trasciatti, Vecchiano (PI) (IT)

(73) Assignee: ABIOGEN PHARMA S.P.A., Ospedaletto (IT)

(21) Appl. No.: 16/616,570

(22) PCT Filed: May 30, 2018
(86) PCT No.: PCT/IB2018/053837

§ 371 Date: Nov. 25, 2019

(30) Foreign Application Priority Data
Jun. 1, 2017 (IT) 102017000060645

(51) Int. Cl. A61K 31/05; A61K 8/34

(52) U.S. Cl. CPC A61K 31/05; A61K 8/347

(57) ABSTRACT

The use of xibornol as an active agent in the treatment of Acne vulgaris is disclosed, said xibornol having shown a remarkable bacteriostatic and bactericidal action on the bacterium mainly responsible of the onset and worsening of Acne vulgaris, i.e. the bacterium Propionibacterium acnes. Pharmaceutical or cosmetic compositions comprising xibornol and suitable pharmaceutically or cosmetically acceptable excipients, for use in the treatment of Acne vulgaris are also disclosed.

### FIELD OF THE INVENTION

The present invention relates to the use of xibornol as an active agent in the treatment of Acne vulgaris.

### BACKGROUND ART

Acne vulgaris is a benign chronic skin disease which affects approximately 80-85% of adolescents and young adults worldwide.

It is characterised by an inflammatory process of the hair follicle and the annexed sebaceous gland thereto. Symptoms are multiple and range from comedones, papules, and pustules, to more destructive manifestations such as nodules, cysts, abscesses, and phlegmons.

Individuals who suffer from this disease, which is aesthetically invasive, also experience considerable discomfort on a psychological level; it has been estimated that, in approximately 15-20% of the most severely affected individuals, psychological discomfort can even develop into serious forms of depression.

The actiology of Acne vulgaris is complex and multifactorial; it is undoubtedly related to the activity of certain specific micro-organisms and is also influenced by other factors, such as lifestyle, personal genetic predisposition, and the general hormonal situation of the individual.

In particular, the bacterium Propionibacterium acnes (Gram positive, facultative anaerobic bacterium) has been proved to play a fundamental role in the development of the inflammatory process of Acne vulgaris.

Indeed, the chemotactic factors induced by Propionibacterium acnes attract monocytes, neutrophils and lymphocytes in the pilosebaceous units, thus stimulating the release of pro-inflammatory molecules.

Furthermore, the bacterium induces the production of sebum by follicles, stimulates the production of pro-inflammatory cytokines such as TNF-α, IL-1β, IL-8 and IL-12, mediated by TOLL-like receptor 2, and produces lipases, proteases and hyaluronidases that contribute to tissue damage.

It has also been observed that often, in addition to the main bacterial action of Propionibacterium acnes, the action of other bacteria has been observed, namely bacteria which are typically non-pathogenic and normally commensal of the human skin but—under pathological conditions of acne—convert into occasional pathogens, which worsen and complicate the course of the disease.

Propionibacterium acnes also has the ability to form a biofilm and this characteristic makes said bacterium particularly resistant to, for example, most of the antibiotic molecules currently available on the market.

Considering thus the multiplicity of symptoms of Acne vulgaris, the complexity of its actiology, with biological mechanisms not yet clearly defined, as well as its evolution strongly dependent also on the specific individual and the lifestyle thereof, doctors are currently using several treatment approaches with the aim to treat the disease.

The most common approaches involve the use of antibiotics administered topically or orally, retinoids administered topically or orally, anti-androgen hormones administered orally, antimicrobials administered topically, keratolytic agents administered topically, combinations of the treatments listed above, as well as, finally, alternative treatments to pharmacological therapies, such as phototherapy, which is often used in combination with the listed above treatments.

Antibiotics most commonly used for the topical treatment of Acne vulgaris include clindamycin and erythromycin.

Those most commonly used for oral administration include erythromycin, tetracycline, doxycycline, minocycline and azithromycin. Sulfamethoxazole, a broad-spectrum antibiotic that has proved to be ineffective when used alone in treating acne, is used in association with trimethoprim.

Tretinoin, isotretinoin, and adapalene are, instead, the most commonly used retinoids for the topical treatment of Acne vulgaris, whereas isotretinoin is the most commonly used retinoid for oral administration.

The most common antimicrobials administered topically include benzoyl peroxide, azelaic acid, and zinc, typically in the form of oxide, acetate, sulfate heptahydrate, picolinate, or gluconate.

Finally, as regards keratolytic agents, the most commonly used include salicylic, glycolic, pyruvic, and trichloroacetic acids.

Given the plurality of available pharmaceutical products, and their different characteristics of action, clinicians have defined different types of treatment approaches depending on both the stage of the disease and the type of individuals to be treated.

The most commonly used treatment approaches, in particular as first-line treatment, include the use of two different active pharmaceutical ingredients, in combination, to be administered topically, which are usually a retinoid in combination with an antimicrobial agent, such as benzoyl peroxide. This type of approach is also preferred, in the first instance, in order to reduce the use of antibiotics and therefore prevent the onset of antibiotic-resistance phenomena.

In cases, though, wherein it is essential to use antibiotics, the general rule is to prescribe these drugs anyway in association with other molecules, for example retinoids or antimicrobials, also in order to reduce the specific dosages of antibiotics.

However, all the treatment agents described above show some limits: antimicrobials have no anti-inflammatory activity, while retinoids have serious side effects including being, in particular, teratogens, and antibiotics do not reduce inflammation and provoke the onset of resistance phenomena.

Therefore, it is felt the need of developing new products which have an antibacterial action, with low potential for resistance induction and, possibly, also exhibit an anti-inflammatory action.

The object of the present invention, therefore, is to find an effective remedy for the treatment of Acne vulgaris, which it is also well tolerated by the organism.

### SUMMARY OF THE INVENTION

Said object has been surprisingly achieved by the use of xibornol as an active agent in the treatment of Acne vulgaris.

In another aspect, the invention relates to the use of xibornol as an antibacterial agent for use in the therapeutic treatment of infections caused by Propionibacterium acnes.

In a further aspect, the present invention relates to a pharmaceutical or cosmetic composition comprising xibornol and suitable pharmaceutically or cosmetically acceptable excipients, for use in the treatment of Acne vulgaris.

### DETAILED DESCRIPTION OF THE INVENTION

The invention therefore relates to the use of xibornol as an active agent in the treatment of Acne vulgaris.

Xibornol, or 3,4-dimethyl-6-isobornylphenol, IUPAC name 4,5-dimethyl-2-[1,7,7-trimethylbicyclo[2.2.1]heptan-2-yl]phenol, is a phenolic derivative of bornane, characterised by the following structural formula:

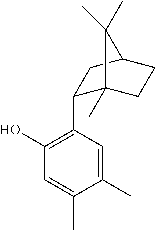 [SMILES: Cc1cc(O)c(C2CC3CCC2(C)C3(C)C)cc1C]

For the purposes of the present invention, the term “xibornol” includes all the optical isomers, geometric isomers, and stereoisomers of 4,5-dimethyl-2-[(1,7,7-trimethylbicyclo[2.2.1]heptan-2-yl]phenol, as well as mixtures thereof, such as mixtures of enantiomers, racemates, and mixtures of diastereoisomers, as well as all polymorphic forms thereof, including amorphous and crystalline forms, co-crystalline forms, as well as anhydrous, hydrated, and solvate forms, pharmaceutically acceptable salts, and mixtures thereof.

In one embodiment of the invention, xibornol is 4,5-dimethyl-2[(1S,2R,4R)-1,7,7-trimethylbicyclo[2.2.1]heptan-2-yl]phenol or 4,5-dimethyl-2-[(1R,2S,4S)-1,7,7-trimethylbicyclo[2.2.1]heptan-2-yl]phenol.

The two single stereoisomers can be obtained by conventional enantiomeric separation techniques; in the examples given below, said stereoisomers were obtained by chromatographic separation using a Chiralpak AD-H, 250×20 mm, 5 μmed chiral column and a 90:10 n-hexane/isopropanol mixture as an eluent.

In another embodiment of the invention, xibornol is a mixture of 4,5-dimethyl-2-[(1S,2R,4R)-1,7,7-trimethylbicyclo[2.2.1]heptan-2-yl]phenol and 4,5-dimethyl-2-[(1R, 2S,4S)-1,7,7-trimethylbicyclo[2.2.1]heptan-2-yl]phenol.

In preferred embodiments, xibornol is a racemate of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol.

As will become evident from the working examples given below, xibornol has been demonstrated to be an active agent usable in the treatment of Acne vulgaris.

In particular, xibornol has shown a remarkable bacteriostatic and bactericidal action on the bacterium mainly responsible of the onset and worsening of Acne vulgaris, i.e. the bacterium Propionibacterium acnes.

Xibornol can therefore be effectively used as an antibacterial agent in the treatment of infections caused by Propionibacterium acnes.

In particular, xibornol can therefore be effectively used as an antibacterial agent in the treatment of infections caused by Propionibacterium acnes, such as progressive macular hypomelanosis and hidradenitis suppurativa (also known as ‘acne inversa’). Furthermore, xibornol has also shown a remarkable anti-inflammatory action in experimental acne models, so that xibornol can also be effectively used as an anti-inflammatory agent in the treatment of Acne vulgaris.

In another aspect, the present invention therefore relates to a pharmaceutical or cosmetic composition comprising xibornol and at least one pharmaceutically or cosmetically acceptable excipient, for use in the treatment of Acne vulgaris.

In a further aspect, the present invention relates to a pharmaceutical or cosmetic composition comprising xibornol and at least one pharmaceutically or cosmetically acceptable excipient, for use in the treatment of infections caused by Propionibacterium acnes.

In an embodiment of the invention, said pharmaceutical or cosmetic composition comprises 4,5-dimethyl-2-[(1S,2R,4R)-1,7,7-trimethylbicyclo[2.2.1]heptan-2-yl]phenol, or 4,5-dimethyl-2-[(1R,2S,4S)-1,7,7-trimethylbicyclo[2.2.1]heptan-2-yl]phenol.

In a further embodiment of the invention, said pharmaceutical or cosmetic composition comprises a mixture of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol.

In a still further embodiment of the invention, said pharmaceutical or cosmetic composition comprises a racemate of 4,5-dimethyl-2-[(1S,2R,4R)-1,7,7-trimethylbicyclo[2.2.1]heptan-2-yl]phenol and 4,5-dimethyl-2-[(1R,2S ,4S)-1,7,7-trimethylbicyclo[2.2.1]heptan-2-yl]phenol.

Said pharmaceutical or cosmetic composition may be administered by external topical, subcutaneous, transdermal, or oral route.

In a preferred embodiment, said pharmacological composition is administered by external topical, subcutaneous, or transdermal route.

When the pharmaceutical or cosmetic composition is to be administrated by external topical, subcutaneous, or transdermal route, it is in the form of a solution, lotion, emulsion, suspension, gel, ointment, cream, paste, spray solution, transdermal patch, wherein the main active ingredient xibornol is suspended or dissolved in one or more excipients.

Examples of excipients suitable for these forms of administration are mineral oil, liquid paraffin, white vaseline, propylene glycol, polyoxyethylene, polyoxypropylene, emulsifying wax, stearyl alcohol, isostearyl alcohol, cetylstearyl alcohol, stearic acid, glyceryl stearate, sodium lauryl sarcosinate, glycerine, diethylene glycol monoethyl ether, polyethylene glycols, polyethylene glycol stearates, starch, hydroxypropyl cellulose, methylcellulose, carbopol, carbomers, methyl paraben, Poloxamer 407, Macrogol 400, purified bentonite, hydroxypropyl methyl cellulose, propyl paraben, myristyl propionate, dimethicone, titanium dioxide, anionic, cationic and non-ionic surfactants, water, and mixtures thereof. Furthermore, the composition may comprise also pH regulators, preservatives, and flavouring agents.

Preferably, the pharmaceutical or cosmetic composition of the invention is to be administered by external topical route.

When the pharmaceutical or cosmetic composition of the invention is to be administrated by external topical, subcutaneous, or transdermal route, in the form of a solution, lotion, emulsion, suspension, gel, ointment, cream, paste, spray solution, or transdermal patch, said composition preferably comprises xibornol in a concentration of 2 μg/mL to 5 mg/mL of composition, more preferably comprises xibornol in a concentration of 4 μg/mL to 5 mg/mL of composition, and even more preferably 9 μg/mL to 2.5 mg/mL.

In a further preferred embodiment, the pharmaceutical composition of the invention is administered by oral route.

When the pharmaceutical or cosmetic composition is to be administered by oral route, said composition is preferably in the form of an orodispersible solid preparation, gel, capsule, tablet, powder, granules, solution, suspension, emulsion, or tincture.

When said pharmaceutical or cosmetic composition is in a tablet or capsule form, examples of particularly suitable excipients are lactose, calcium phosphate, microcrystalline cellulose, ethyl cellulose, dextrose, fructose, mannitol, sorbitol, sucrose, xylitol, starch, pregelatinised starch, sodium carboxymethylcellulose, gelatin, hydroxypropyl cellulose, hydroxypropyl methylcellulose, methylcellulose, povidone, sodium alginate, magnesium stearate, stearic acid, talc, colloidal silica, and mixtures thereof.

When said pharmaceutical or cosmetic composition is in the form of an aqueous suspension, examples of particularly suitable excipients are glycerine, polyethylene glycol, microcrystalline cellulose, xanthan gum, water, emulsifying and resuspending agents, in addition to sweetening agents such as sucrose, sodium saccharin, aspartame, sodium cyclamate, preservatives, pH regulators, antioxidants, flavourings, colourants, and mixtures thereof.

The pharmaceutical or cosmetic composition of the invention to be administered by oral route in the form of an orodispersible solid preparation, gel, capsule, tablet, powder, granules, solution, suspension, emulsion, or tincture, preferably comprises at least a unit dose of xibornol ranging from 10 mg to 500 mg, preferably at least a unit dose of xibornol of 200 mg to 300 mg, and even more preferably at least a unit dose of 250 mg of xibornol. In a still further preferred embodiment of the invention, the pharmaceutical or cosmetic composition of the invention to be administered by oral route in the form of a solution, suspension, emulsion, gel, or tincture, comprises xibornol in a concentration of 10 mg/mL to 35 mg/mL, preferably in a concentration of 20 to 30 mg/mL.

All the above described pharmaceutical compositions may be prepared by using methods known in the art depending on the administration route.

### EXPERIMENTAL PART

### Example 1

### Evaluation of the Efficacy of Xibornol Against Propionibacterium acnes

The following experimental part contains the results of a study conducted to demonstrate the antibacterial properties of xibornol, and more specifically those of a racemate of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol, against Propionibacterium acnes.

In order to perform the antimicrobial tests in aqueous solutions (broth and agar), said xibornol, a lipophilic molecule, was dissolved in a hydrophilic solvent, and more specifically in isopropanol.

### Solvent Toxicity Test on Propionibacterium acnes

The possible effect of different concentrations of solvent used to dissolve the xibornol on the growth of Propionibacterium acnes was assessed.

The test was performed on 96-well plates wherein the bacterium was added, in a final concentration of 5×10⁵ bacteria/mL, to the culture medium in each well. This final concentration was obtained by appropriately diluting the McFarland 0.5 standard.

For comparison with the positive controls devoid of isopropanol, the test showed that, when using 4% isopropanol, capable of dissolving up to 1200 μg/mL of xibornol, there were no detectable effects on the growth of the Propionibacterium acnes due to the solvent.

### Determination of the Minimum Inhibitory Concentration (MIC) and the Minimum Bactericidal Concentration (MBC) Active on Propionibacterium acnes

Two strains of Propionibacterium acnes were used for this test: the Grerath strain (ATCC-11827) and the VPI 0389 strain (ATCC-6919).

Clostridial differential broth (CDB, liquid medium) and reinforced clostridial agar (RCA, solid medium) were used for the culture. Growth occurred under anaerobic conditions obtained in a 7.0 L GENBOX JAR.

The anaerobic conditions were obtained by activating the Anaerocult A preparation and the anaerobiosis control was assessed by using ANAEROTEST strips for microbiology.

The Propionibacterium acnes was incubated at 37° C. for 72 hours.

Propionibacterium acnes strains were reactivated from ATCC vials, rehydrated with CDB medium, and then seeded on RCA plates.

To cultivate Propionibacterium acnes in broth, isolated colonies were taken from culture plates and inoculated in the CBD medium.

After culturing, the concentration of bacterial cells was normalised using the McFarland 0.5 standard.

A 96-well plate was prepared in order to establish the minimum inhibitory concentration; the xibornol was dissolved in a 4% isopropanol solution in various concentrations, and the samples were prepared according to the experimental plan shown in the following table 1.

TABLE 1

| 1 | 2 | 3 | 4 | 5 | 6 | 7 | 8 | 9 | 10 | 11 | 12 | |
|---|---|---|---|---|---|---|---|---|---|---|---|---|
| 1200 | 600 | 300 | 150 | 75 | 37.5 | 18.75 | 9.37 | 4.69 | 2.34 | 1.17 | 0 | A |
| μg/mL | μg/mL | μg/mL | μg/mL | μg/mL | μg/mL | μg/mL | μg/mL | μg/mL | μg/mL | μg/mL | μg/mL | |
| 1200 | 600 | 300 | 150 | 75 | 37.5 | 18.75 | 9.37 | 4.69 | 2.34 | 1.17 | 0 | B |
| μg/mL | μg/mL | μg/mL | μg/mL | μg/mL | μg/mL | μg/mL | μg/mL | μg/mL | μg/mL | μg/mL | μg/mL | |
| 1200 | 600 | 300 | 150 | 75 | 37.5 | 18.75 | 9.37 | 4.69 | 2.34 | 1.17 | 0 | C |
| μg/mL | μg/mL | μg/mL | μg/mL | μg/mL | μg/mL | μg/mL | μg/mL | μg/mL | μg/mL | μg/mL | μg/mL | |
| 1200 | 600 | 300 | 150 | 75 | 37.5 | 18.75 | 9.37 | 4.69 | 2.34 | 1.17 | 0 | D |
| μg/mL | μg/mL | μg/mL | μg/mL | μg/mL | μg/mL | μg/mL | μg/mL | μg/mL | μg/mL | μg/mL | μg/mL | |
| 1200 | 600 | 300 | 150 | 75 | 37.5 | 18.75 | 9.37 | 4.69 | 2.34 | 1.17 | 0 | E |
| μg/mL | μg/mL | μg/mL | μg/mL | μg/mL | μg/mL | μg/mL | μg/mL | μg/mL | μg/mL | μg/mL | μg/mL | |
| 1200 | 600 | 300 | 150 | 75 | 37.5 | 18.75 | 9.37 | 4.69 | 2.34 | 1.17 | 0 | F |
| μg/mL | μg/mL | μg/mL | μg/mL | μg/mL | μg/mL | μg/mL | μg/mL | μg/mL | μg/mL | μg/mL | μg/mL | |
| 0% IP | 0% IP | 0% IP | | | | | | | | | | G |
| 0% IP | 0% IP | 0% IP | | | | | | | | | | H |

All the wells in rows D, E, F and the first three wells from the left in row H contained Propionibacterium acnes at the final concentration of 5×10⁵ bacteria/mL. Rows A, B, and C did not contain any bacterial culture and were the negative controls of each triplicate of wells in the corresponding rows D, E, F.

Row G (wells 1, 2, 3) was the negative control and contained only the culture medium, without bacteria, xibornol, or isopropanol.

Row H (wells 1,2,3) was the positive control and contained the bacterial culture without xibornol or isopropanol.

Unmarked wells were empty.

The reading of the plate, after an incubation under anaerobic conditions at 37° C., was performed after 72 hours.

A first spectrophotometer reading was taken, including measurement of the OD (optical density) at 600 nm.

To highlight the presence or absence of bacterial growth on the plate described above, PrestoBlue was also used, which is a resazurin-based solution which uses the reducing power of cells to measure cell proliferation. Said solution is blue and turns into red upon contact with live bacterial cells.

After having incubated the plate with the PrestoBlue and read the fluorescence values by using a Victor 3 Wallac microplate reader, the data were confirmed.

### Results

The wells where bacterial growth was observed (red colour) were wells 9, 10, 11, 12 in rows D, E, F, i.e. those wells in which xibornol was present in concentrations of 4.69 μg/mL, 2.34 μg/mL, 1.17 μg/mL and 0μg/mL, respectively.

As expected, bacterial growth was also observed in wells 1, 2 and 3 in row H (positive controls containing the bacterial culture only).

The minimum inhibitory concentration (MIC) turned out to be 9.37 μg/mL.

The test was repeated three times and the results were always superimposable.

To determine the minimum bactericidal concentration (MBC), 10 μl of solution was taken from each well on the above described plate and inoculated on RCA plates containing the same concentrations of xibornol in 4% isopropanol present in the wells of the plate.

Also in this case, it was observed that no growth (MBC) occurred with the same concentration previously referred to as the minimum inhibitory concentration (MIC), i.e. 9.37 μg/mL, while with the 4.69 μg/mL concentration, there was massive growth, comparable to that of the positive control, suggesting that the MIC and the MBC should range from 4.69 μg/mL to 9.37 μg/mL.

This last experiment was also replicated three times, providing perfectly superimposable results.

The studies carried out therefore demonstrated, very effectively, the antibacterial properties of xibornol against Propionibacterium acnes, thereby confirming the possibility of using xibornol in the treatment of infections caused by Propionibacterium acnes, such as, in particular, Acne vulgaris.

### Example 2

### Assessment of Skin Tolerability of Xibornol

In order to establish the usable concentrations of xibornol in solution for the production of pharmaceutical forms intended for external topical use, a skin irritation and sensitisation test was carried out, according to standard OECD TG 404, method B.4, Annex V, Directive 67/548/EEC.

A RHE EPISKIN artificial epidermis unit was used for the test. The kit consisted of 24 reconstructed epidermis units with a total area of 0.33 cm².

Each unit consisted of a collagen matrix with a stratified, differentiated epidermis derived from human keratinocytes placed on top thereof.

The substances to be tested were placed in contact (42′) with the epidermis and the effects assessed after 42 hours, incubating the units with MTT (3-(4,5-dimethylthiazol-2-yl)-2,5-diphenyltetrazolium bromide) which, under the experimental conditions, reduced to formazan upon contact with metabolically active cells, turning the coloured solution from yellow into blue. Since the obtained colour is directly proportional to the concentration of formazan, and therefore to the cell viability, after incubation with MTT for 3 hours, optical density readings were taken by using the spectrophotometer (DO 572-650 nm).

### Results

The experimental tests carried out showed that the liquid formulations containing xibornol, isopropanol, and PBS buffer (phosphate buffered saline), comprising a concentration of xibornol less than 5 mg/mL and, even more preferably, less than 2.5 mg/mL, may be administered topically without causing any irritation or sensitisation of the epidermis.

### Example 3

### Preparation of a Pharmaceutical Composition in the Form of a Lotion Comprising a Racemate of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol

100 g of a topical lotion was prepared containing xibornol in the form of a racemate of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol.

The lotion comprised, in particular, the ingredients listed below in Table 2:

TABLE 2

| | Amount (in g) per |
|---|---|
| Component | 100 g of lotion |
| Xibornol | 0.3 |
| Purified bentonite (Polargel NF) | 4 |
| Hydroxypropyl methylcellulose | 1 |
| Methylparaben | 0.2 |
| Propylparaben | 0.2 |
| Glyceryl stearate | 2 |
| Propylene glycol | 6 |
| Myristyl propionate | 2 |
| Dimethicone | 0.5 |
| Titanium dioxide | 1 |
| Purified water | balance to 100 g |

### Preparation

The Polargel NF was added to approximately 30 g of water, rapidly stirred and then left to hydrate for 15 minutes.

The obtained mixture was filtered with a large mesh sieve, the hydroxypropyl methylcellulose was added, and the mixture was mixed until devoid of lumps. Next, the parabens were added, under stirring, and heated to approximately 90° C., until their complete dissolution.

Separately, methylparaben, propylparaben, glyceryl stearate, propylene glycol, myristyl propionate, dimethicone and hydroxypropyl methylcellulose were mixed, in the amounts reported in the table, in approximately 50 g of water.

This second mixture was added to the first one containing Polargel, hydroxypropyl methylcellulose and parabens, while mixing well.

Finally, the titanium dioxide and xibornol were added to said last mixture, under stirring. The preparation thus obtained, in the form of a lotion, could be directly applied, topically, for the treatment of Acne vulgaris.

### Example 4

### Preparation of a Pharmaceutical Composition in the Form of a Gel Comprising a Racemate of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol

100 g of a topical gel containing xibornol in the form of a racemate of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol were prepared.

The gel comprised, in particular, the ingredients listed below in Table 3:

TABLE 3

| | Amount (in g) per |
|---|---|
| Component | 100 g of gel |
| Xibornol | 0.3 |
| Macrogol 400 | 20 |
| Propylene glycol | 20 |
| POLOXAMER 407 | 20 |
| Purified water | balance to 100 g |

### Preparation

POLOXAMER 407 was dissolved in a solution of xibornol, Macrogol 400, and propylene glycol, heated to approximately 70° C., then mixed with purified water and cooled until the air bubbles were completely eliminated. The resulting gel may be administered for topical use in the treatment of patients suffering from Acne vulgaris.

### Example 5

### Preparation of a Pharmaceutical Composition in the Form of an Ointment Comprising a Racemate of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol

100 g of a topical ointment containing xibornol in the form of a racemate of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol were prepared.

The ointment comprised, in particular, the ingredients listed below in Table 4:

TABLE 4

| | Amount (in g) per |
|---|---|
| Component | 100 g of ointment |
| Xibornol | 0.3 |
| Liquid paraffin | 3 |
| White vaseline | balance to 100 g |

### Preparation

The liquid paraffin and white vaseline were heated to 115° C. and the temperature was maintained for at least 3 hours. The mixture was then mixed and subsequently cooled to a temperature of 40-45° C.

A small portion of this mixture was put aside.

Xibornol was added to the remaining part, stirred until complete dispersion, and then sieved with a 74 micron sieve; the portion of the paraffin and vaseline mixture previously set aside was also sieved.

The resulting mixture was mixed for 2 hours and subsequently subjected to slow cooling to prevent condensation phenomena.

The cooled ointment thus obtained was ready to be used for topical use in the treatment of patients suffering from Acne vulgaris.

### Example 6

### Study of the Anti-Inflammatory Efficacy of Topical Applications of Xibornol in a Mouse Model for Acne vulgaris

The aim of the present study was to demonstrate the anti-inflammatory efficacy of xibornol on Acne vulgaris induced by Propionibacterium acnes by monitoring the microbial inflammatory effects thereof.

For the experiment, female Balb/c strain mice were used, grouped according to the experimental plan shown below in Table 5:

TABLE 5

| | N. of individuals | |
|---|---|---|
| Group | per group | Treatment |
| Sham | 3 | Untreated |
| Control | 6 | Carrier |
| Treated group | 6 | 0.1% xibornol |
| | | (0.79 mg/mL) |

The sham group consisted of mice which received an intradermal injection, in each ear, of 25 μl of D-PBS (Dulbecco's phosphate buffered saline), while the other two groups consisted of mice which received an intradermal injection, in each ear, of 25 μl of D-PBS containing 10⁸ CFU (colony-forming units) of Propionibacterium acnes.

As of the day following the injection:
the sham group of mice was not treated;
the control group was treated, once a day, for 7 consecutive days, directly to the sites of inoculation of the bacterium, with a topical application of the formulation without active principle, i.e. with the carrier, consisting of three parts of paraffin oil and two parts of filamentary vaseline;
the control group was subjected, once a day, for 7 consecutive days, directly to the sites of inoculation of the bacterium), with a topical application of the formulation containing xibornol in a concentration of 0.1% (corresponding to 0.79 mg/mL) in the carrier, consisting of three parts of paraffin oil and two parts of filamentary vaseline.

The mice were sacrificed 24 hours after the last topical application.

The anti-inflammatory effects of xibornol treatment were verified by determining, on the ears removed from the mice during necropsy, the levels of inflammatory cytokine IL-8 in the ear homogenate supernatants.

### Results

The lesions present in the ears of the mice were removed surgically and weighed, after discarding all the surrounding tissues.

Dissection of the selected material was performed following the instructions given in the Epidermidis dissociation Kit Mouse supplied by MACS Milteny Biotech (code n. 130-095-928).

Each sample was transferred to a well on a 6-well plate containing 2 mL of Dulbecco phosphate buffered saline (D-PBS) with enzyme G and incubated at 4° C. for sixteen hours.

The following day, the samples were chopped using scalpels and tweezers, transferred into tubes with 3.9 mL of buffer S containing enzyme P and enzyme A and incubated at 37° C. for twenty minutes. Enzymatic activities were then interrupted by adding 4 mL of PB buffer. The tubes were then subjected to mechanical stirring in order to optimise disintegration.

Subsequently, the lysate was filtered through a 70 μm filter and the eluate was collected in a 50 mL tube. The filter was then washed with 10 mL of PB buffer and, finally, the tubes were centrifuged at room temperature at 3300×g for 20 minutes.

Finally, 4.5 mL of the supernatants of each sample were collected and stored at a temperature of −80° C. for assaying cytokines.

The cytokine IL-8 assay was performed by using commercial kits for ELISA (enzyme-linked immunosorbent assay) and following the manufacturer's instructions.

The IL-8 assay results (average concentrations expressed as pg/mL) are shown below in Table 6.

TABLE 6

| | | | Treated group |
|---|---|---|---|
| | Sham | Control | 0.1% xibornol |
| Average concentration | 206.44 | 221.72 | 190.61 |
| of IL-8 (pg/mL) | | | |

As it can be seen from the results shown in Table 6, the application of 0.1% xibornol resulted in a significant reduction in IL-8 levels with respect to the control group, thereby demonstrating the anti-inflammatory properties of xibornol.

### CONCLUSIONS

In conclusion, all the tests above described demonstrate that xibornol is effective on Propionibacterium acnes as a bacteriostatic and bactericidal agent, with an experimental minimum inhibitory concentration (MIC) value being coincident with the value of the minimum bactericidal concentration (MBC), both falling within a range of 4.69 μg/mL to 9.37 μg/mL.

Xibornol is therefore an effective antibacterial agent, whose efficacy is even comparable to that of clindamycin, the well-known antibiotic currently used in the treatment of Acne vulgaris and may be administered topically in pharmaceutical or cosmetic forms which comprise xibornol in concentrations in the range of 4 μg/mL to 5 mg/mL and, more preferably, in the range of 9 μg/mL to 2.5 mg/mL.

Furthermore, in vivo tests have also demonstrated the anti-inflammatory action of xibornol. Xibornol is therefore an effective active antibacterial and anti-inflammatory agent usable in the treatment of Acne vulgaris.

More generally, the tests performed have demonstrated that xibornol is an effective antibacterial and anti-inflammatory agent in infections caused by Propionibacterium acnes and it is therefore usable in the treatment of any disease caused by Propionibacterium acnes, including, in particular progressive macular hypomelanosis and hidradenitis suppurativa (also known as ‘acne inversa’).

Conveniently, said xibornol may also be formulated as a pharmaceutical preparation having the form of a lotion, gel, or ointment, for topical administration in patients suffering from Acne vulgaris, progressive macular hypomelanosis, or hidradenitis suppurativa.

1. A method for the treatment of Acne vulgaris, the method comprising the step of administering of a therapeutically effective amount of xibornol, as a virucidal agent, to patients in need thereof.

2. The method of claim 1, wherein xibornol also acts as an active antibacterial agent in the treatment of Acne vulgaris.

3. The method of claim 1, wherein xibornol also acts as an active anti-inflammatory agent in the treatment of Acne vulgaris.

4. A method for the treatment of infections caused by Propionibacterium acnes, the method comprising administering a therapeutically effective amount of xibornol, as an antibacterial agent, to patients in need thereof.

5. The method of claim 1, wherein said xibornol is 4,5-dimethyl-2-[(1S,2R,4R)-1,7,7-trimethylbicyclo[2.2.1]heptan-2-yl]phenol or 4,5-dimethyl-2-[(1R,2S,4S)-1,7,7-trimethylbicyclo[2.2.1]heptan-2-yl]phenol.

6. The method of claim 1, wherein said xibornol is a mixture of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol.

7. The method of claim 6, wherein said xibornol is a racemic mixture of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol.

8. The method of claim 1, comprising the step of administering a therapeutically effective amount of a pharmaceutical or cosmetic composition comprising xibornol and at least one pharmaceutically or cosmetically acceptable excipient to patients in need thereof.

9. The method of claim 4, comprising the step of administering a therapeutically effective amount of a pharmaceutical or cosmetic composition comprising xibornol and at least one pharmaceutically or cosmetically acceptable excipient to patients in need thereof.

10. The method of claim 8, wherein the pharmaceutical or cosmetic composition is administered via external topical, subcutaneous, transdermal or oral route.

11. The method of claim 10, wherein said pharmaceutical or cosmetic composition is administered via external topical route.

12. The method of claim 11, wherein said pharmaceutical or cosmetic composition is in the form of a solution, lotion, emulsion, suspension, gel, ointment, cream, paste, spray solution, or transdermal patch.

13. The method of claim 11, comprising xibornol at a concentration of 2 μg/mL to 5 mg/mL of composition.

14. The method of claim 10, wherein said pharmaceutical or cosmetic composition is administered via oral route.

15. The method of claim 14, wherein said pharmaceutical or cosmetic composition to be administered via oral route is in the form of an orodispersible solid preparation, gel, capsule, tablet, powder, granules, solution, suspension, emulsion, or tincture.

16. The method of claim 14, said pharmaceutical or cosmetic composition comprising at least a unit dose of xibornol of 10 mg to 500 mg.

17. The method of claim 14, wherein said pharmaceutical or cosmetic composition to be administered via oral route is in the form of a solution, suspension, emulsion, gel or tincture.

18. The method of claim 17, comprising xibornol at a concentration of 10 mg/mL to 35 mg/mL.

19. The method of claim 4, wherein said xibornol is 4,5-dimethyl-2-[(1S,2R,4R)-1,7,7-trimethylbicyclo[2.2.1]heptan-2-yl]phenol or 4,5-dimethyl-2-[(1R,2S,4S)-1,7,7-trimethylbicyclo[2.2.1]heptan-2-yl]phenol.

20. The method of claim 4, wherein said xibornol is a mixture of 4,5-dimethyl-2-[(1S,2R,4R)-1,7,7-trimethylbicyclo[2.2.1]heptan-2-yl]phenol and 4,5-dimethyl-2-[(1R,2S,4S)-1,7,7-trimethylbicyclo[2.2.1]heptan-2-yl]phenol.
